(19) United States
(12) Patent Application Publication
(10) Pub. No.: US 2019/0183758 A1
(43) Pub. Date: Jun. 20, 2019

(54) LIQUOR-BASED UNDERARM DEODORANT

(71) Applicants: Erica Feucht, Loveland, CO (US); Jason Feucht, Loveland, CO (US)

(72) Inventors: Erica Feucht, Loveland, CO (US); Jason Feucht, Loveland, CO (US)

(21) Appl. No.: 15/848,981

(22) Filed: Dec. 20, 2017

(51) Int. Cl. A61K 8/34; A61K 8/26; A61K 8/9789; A61Q 15/00

(52) U.S. Cl. CPC A61K 8/34; A61K 8/26; A61K 2800/56; A61Q 15/00; A61K 8/9789

(57) ABSTRACT

An underarm deodorant has a solvent, an antiseptic component, and a potable alcoholic component having a distinctive fragrance, taste, or both. The underarm deodorant can also include an antiperspirant component and a chemical pheromone. The antiseptic and alcoholic components can be encapsulated in time-release polymer enclosures. The alcohol component can be in the form of beer, wine, whiskey, vodka, brandy, vermouth, bitters, champagne, anise, aperitifs, absinthe, scotch, rye, gin, rum, bourbon, tequila, and other liquors, cognac, or liqueurs combined to create not only the odor of such liquors, but taste as well.

### FIELD OF THE INVENTION

This invention relates to deodorants and, more particularly, to a potable, liquor-based underarm deodorant.

### BACKGROUND OF THE INVENTION

Human skin is naturally populated with numerous micro-organisms. These organisms are nourished by various skin secreted substances, skin cell debris, breakdown products of the skin and the organisms themselves. The “skin secretions” are eccrine and apocrine sweat, and lipid-soluble sebum. Eccrine sweat is normally odorless and remains odorless after secretion, although odoriferous food and drug substances may be excreted with it. Apocrine glands are normally associated with hair follicles and are confined mainly to the groin, perianal, areola and armpits. They produce a scanty, milky substance that is odorless upon secretion, but becomes odoriferous upon bacterial decomposition. Apocrine glands are considered to be a primary contributor for malodor. The sebaceous glands are distributed over the skin surface except the palms and dorsae. They are most numerous on the scalp, forehead, face, back, and chest. The secretion, sebum, consists mainly of fatty materials, wax esters, cholesterol and its esters, and squalene.

Body odor is most commonly caused by fatty acids on skin and from malodors from bacterial sources. The unpleasant odors are mainly organic molecules which have different structures and functional groups, such as amines, acids, alcohols, aldehydes, ketones, phenolics, polycyclics, indoles, aromatics, polyaromatics, etc. They can also be made up of sulfur-containing functional groups, such as, thiol, mercaptan, sulfide and/or disulfide groups.

Odor causing bacteria and fungi often flourish in warm, moist conditions, particularly where they have easy access to nourishment such as skin secretions and skin cell debris. Accordingly, attempts have been made to control odor through moisture absorption, depriving the bacteria responsible for body odor of the moist/humid environment they need to proliferate and grow. Such efforts include the use of powders and/or antiperspirants. Powders and powder-based compositions may be difficult to apply and have limited absorption capabilities. Therefore, use of body powders is generally undesirable and/or ineffective for day to day body odor control for the entire body. Antiperspirants are not useful in a body odor control product for use over the entire body as they may interfere with the body's thermal regulatory process by inhibiting perspiration through the action of astringent salts. Additionally, such salts may be irritating to a large number of users, particularly when applying them to sensitive areas such as the pelvic region.

Known deodorants attempt to control odor through a variety of means. Deodorants may include antibacterial compounds which help destroy and/or control the amount of bacteria present on the skin, thereby minimizing odor produced via bacterial metabolism of the skin secretions.

Some 100 years ago, deodorants and antiperspirants were invented to mask human body odor. The first patents for deodorants were filed in the 1860s, when inventors used sanitizing agents such as ammonium chloride, sodium bicarbonate, and formaldehyde to kill microorganisms. Zinc oxide, still commonly found in deodorants, was one of the first ingredients in the early deodorants. Current deodorant ingredients vary vastly, from antibiotics to citronella oil. All are intended to control bacterial growth.

Prior to those inventions, people washed themselves and/or used fragrances to accomplish this odor masking. Ancient Egyptians had perfumes specifically intended to cover human body odor. But formulas to block odor have only been in wide use for only a century.

Over the years, many perfumes have been used to mask body odors. Not only are such perfumes often inadequate at fully concealing the body odors, very often they are irritating to the user's skin. Additionally, the perfume odor itself may be irritating or offensive to the user's respiratory system and/or olfactory senses, as well as to nearby individuals.

Deodorants and antiperspirants differ from perfumes because they not only mask human body odors, they thwart its production. Deodorants typically include antiseptic ingredients that kill or disable bacteria living in the armpit. That region's warm and moist environment is an optimal habitat for corynebacteria and other sweat-eating microorganisms. These life-forms generate malodorous waste by-products such as trans-3-methyl-2-hexenoic acid, the compound responsible for the characteristic odor of human sweat.

Whereas deodorants attempt to kill the odor-producing microorganisms, antiperspirants block sweat pores, preventing the flow of nutrients to armpit microorganisms. Both products typically include a scent, usually in the form of perfume. Deodorants work by neutralizing the smell of the sweat and by antiseptic action against bacteria. Deodorants do not interfere with sweating, a natural cooling process, as do antiperspirants.

The array of active ingredients in deodorant contrasts with those found in antiperspirants. Antiperspirants have always relied on aluminum salts, such as aluminum chloride, to stop the flow of sweat. More recently, antiperspirant manufacturers have used aluminum formate, aluminum chlorohydroxide, and aluminum zirconium tetrachlorohydrate.

Aside from active ingredients, the way that deodorants and antiperspirants are applied to the armpit has evolved since their inception. The same holds true for the solvents in which the active ingredients are dissolved or suspended. Early antiperspirants suspended their active ingredient in alcohol or, more often, acids, and had to be swabbed with cotton balls on the armpit. These formulations were slow to dry and prone to irritating skin and ruining clothing. Likewise, early deodorants were often sold as creams, which were rubbed by hand on the armpit, often leaving these zones and clothing greasy. The roll-on deodorant was an application mechanism invented in the 1940s. Sticks, gels, and aerosols emerged in the decades thereafter.

Although these relatively new deodorant and antiperspirant products and application or delivery mechanisms improved the aesthetics and ease of application, the biggest nonactive ingredient innovation came in the 1970s, when quick-dry cyclomethicones, also known as methylsiloxanes, were used. Cyclomethicone solvents are now widespread in many antisweat products because they do not irritate skin and they evaporate quickly, leaving behind the deodorant or antiperspirant's active ingredient, but no greasiness or stickiness.

Deodorant compositions have been described in the chemical and cosmetic literature, generally in the form of emulsion sticks, but also may be aerosols, roll-ons, pads, pump sprays, and even soap bars. In recent decades, makers of deodorants and antiperspirants began to encapsulate active ingredients in time-release polymer enclosures so the products last longer.

Common chemicals in deodorants are purified water, guar gum, vegetable glycerin, glycerol stearate, patchouli, sandalwood essential oil, and lavender. Purified water is an active absorber, which, when it comes into contact with air, absorbs carbon dioxide, making it acidic. Guar is digestive fiber used in the food industry as a thickening agent. It helps control the rate of the process absorbing of nutrients, including sugar, spreading it over a longer period of time. Vegetable glycerin can be added to increase moisturizing qualities. Glycerol stearate is a smooth, white powder with odor and taste. Patchouli is fresh oil used in skin care when cracked, dry, itchy, or mature. It is strengthening oil often used as a fixative for perfume blends. Sandalwood essential oil is used for perfumery, cosmetics, and aromatherapy. Lavender is moisturizing to skin.

Coupled with the goal of masking or eliminating body odor, modern perfumes, scents, body washes, after-shave lotions, balms, and gels have been produced to increase sexual magnetism between the wearer of such products and those they wish to influence or entice. It has been found that both the scent and taste of certain alcoholic beverages can act as an intoxicant or an aphrodisiac. Moreover, pheromones have been used in various products with similar results. Along these lines, the growing field of aromatherapy is based on using natural substances to enhance psychological and physical well-being.

It is with this realization that the present inventors have formulated deodorants having alcoholic fragrances and tastes. These formulations can have the effect of heightening erotic stimulation for parties in intimate situations.

### DESCRIPTION OF RELATED ART

U.S. Pat. No. 9,511,034 issued to Garrett for METHOD FOR APPLYING A SKIN TREATMENT issued on Dec. 6, 2016, describes a skin treatment for managing skin conditions ranging from scars, including keloids, to burns and wrinkles. The skin treatment may comprise several components including a cleansing solution, a silicone elastomer, and a mineral powder that may be utilized to treat and camouflage the affected skin. In one embodiment, a cleansing component is used to prime a skin area, cleansing and treating the area so that a silicone component can heal the skin.

U.S. Pat. No. 9,789,038 issued to Fan, et al. for ANTIPERSPIRANT/DEODORANT COMPOSITIONS issued on Oct. 17, 2017, describes a composition comprising at least one active chosen from at least one antiperspirant active and at least one deodorant active; a first gellant chosen from at least one fatty alcohol and at least one hydrocarbon of the formula CₙH₂ₙ₊₂, wherein n is about 20 to about 100, and the hydrocarbon is at least 90% linear; at least one soybean oil having an iodine value of greater than 0 to about 20; and at least one silicone.

U.S. Pat. No. 4,781,917 issued to Luebbe, et al. for ANTIPERSPIRANT GEL STICK issued on Nov. 1, 1988, describes antiperspirant gel stick compositions substantially free of unbound water comprising from about 5 to about 50% of a solubilized antiperspirant active, from about 7% to about 35% of intermediate polarity emollients, from about 1% to about 5% of a benzylidene sorbitol, from about 15% to about 75% of a polar solvent, from about 1% to about 20% of a coupling agent and from about 0.5% to about 10% of a buffering agent. Also described is a method for treating or preventing perspiration and malodor associated with human underarm perspiration.

### SUMMARY OF THE INVENTION

In accordance with the present invention, there is provided an underarm deodorant that has a solvent, an antiseptic component, and an alcoholic component having a distinctive fragrance, taste, or both. The underarm deodorant can also include an antiperspirant component and a chemical pheromone. The antiseptic and alcoholic components can be encapsulated in time-release polymer enclosures. The alcohol component can be in the form of beer, whiskey, vodka, brandy, vermouth, bitters, champagne, anise, aperitifs, absinthe, scotch, rye, gin, rum, bourbon, and other liquors, cognac, and liqueurs combined to create not only the odor of such liquors, but taste as well.

It is therefore an object of the invention to provide a deodorant having a scent and/or taste of an alcoholic beverage.

It is a further object of the present invention to provide an antiperspirant having a scent and/or taste of an alcoholic beverage.

It is a further object of the present invention to provide one or both of the aforementioned products additionally having a scent and/or taste of cannabis.

It is a further object of the present invention to provide one or both of the aforementioned products additionally having a pheromone component.

These and other objects and advantages of the present invention are more readily apparent with reference to the following detailed description and the accompanying drawing.

### BRIEF DESCRIPTION OF THE DRAWINGS

A complete understanding of the present invention may be obtained by reference to the accompanying drawing, when considered in conjunction with the subsequent detailed description, in which:

FIG. 1 is a high level flow chart of the steps used to produce the liquor-based deodorant in accordance with the present invention.

### DETAILED DESCRIPTION OF THE PREFERRED EMBODIMENT

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

The underarm deodorant has a solvent, an antiseptic component, and an alcoholic component having a distinctive fragrance, taste, or both. The alcohol component can be a liquor in the form of beer, wine, whiskey, vodka, brandy, vermouth, bitters, champagne, anise, aperitifs, absinthe, scotch, rye, gin, rum, bourbon, tequila, and other liquors, cognac, and liqueurs combined to create not only the odor of such liquors, but taste as well.

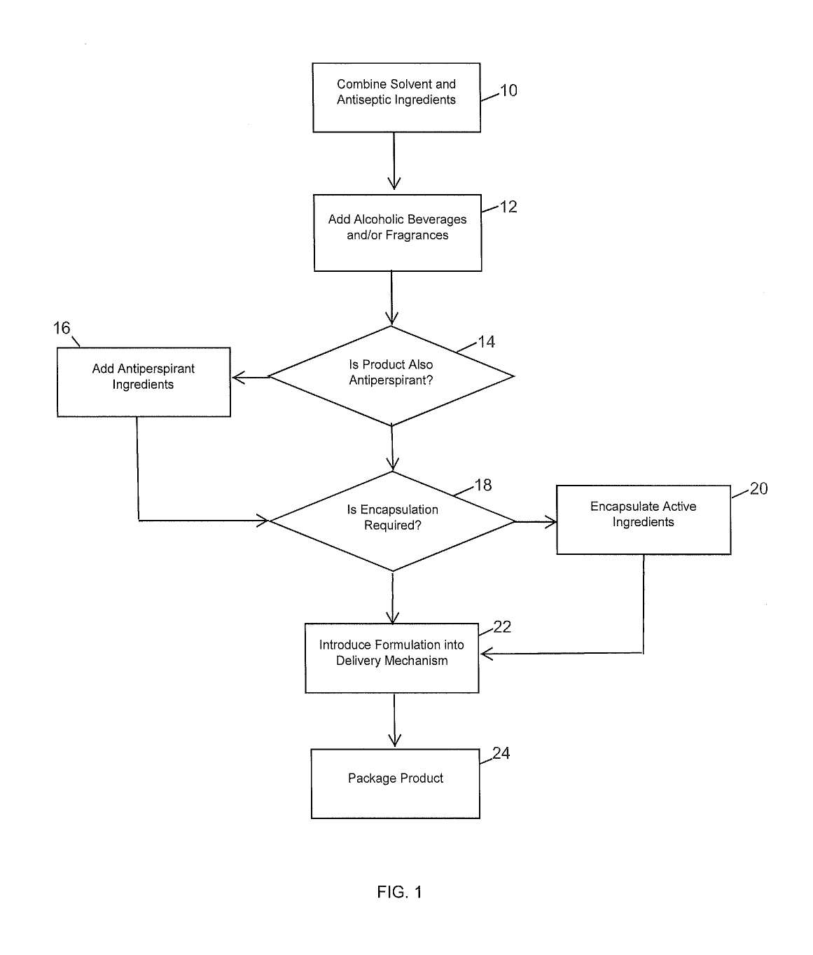

Referring now to FIG. 1, there is shown a flow chart of the steps used to produce the inventive, liquor-based deodorant in accordance with the present invention. The first step in producing the deodorant is combining: a solvent, such as one that includes an antiseptic, such as an antibacterial compound, purified water, guar gum, vegetable glycerin, glycerol stearate, patchouli, and/or sandalwood essential oil, step 10. An alcoholic beverage or fragrance thereof is then added, step 12. The potable alcohol or scent thereof can be beer, wine, whiskey, vodka, brandy, vermouth, bitters, champagne, anise, aperitifs, absinthe, scotch, rye, gin, rum, bourbon, tequila, and other liquors, cognac, and liqueurs, or a combination of some of those beverages. Other liquors or liqueurs may also be used and are considered to be within the scope of the present invention. If the product is also an antiperspirant, step 14, the antiperspirant ingredients can be added, step 16. The antiperspirant ingredients should include at least one antiperspirant component that preferably includes an aluminum salt.

If encapsulation of the active ingredients is required, step 18, such active ingredients are then encapsulated in time-release polymer enclosures, step 20.

The aforementioned ingredients of steps 10, 12, and 16 are then formed into a delivery mechanism, such as a stick, gel, aerosol, pad, pump spray, or soap bar, step 22. Other delivery mechanisms may also be devised without exceeding the scope of the present invention.

The delivery mechanism is then packaged in a convenient form for consumers, step 24, that includes space for identifying the product with an appropriate trademark, list of ingredients, warnings, and method of application.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

1-16 (canceled)

17. An underarm deodorant, comprising:
a) a solvent;
b) an antiseptic component; and
c) an alcoholic component.

18. The underarm deodorant in accordance with claim 17, further comprising:
d) an antiperspirant component.

19. The underarm deodorant in accordance with claim 18, further comprising:
e) a chemical pheromone.

20. The underarm deodorant in accordance with claim 17, wherein at least one of the antiseptic and alcoholic components (b)-(c) is encapsulated in time-release polymer enclosures.

21. The underarm deodorant in accordance with claim 18, wherein at least one of said antiseptic, alcoholic, and antiperspirant components (b)-(d) is encapsulated in time-release polymer enclosures.

22. The underarm deodorant in accordance with claim 17, wherein the product is in the form selected from a set of application mechanisms consisting of: emulsion sticks, aerosols, roll-ons, pads, pump sprays, and soap bars.

23. The underarm deodorant in accordance with claim 17, wherein the antiperspirant comprises an aluminum salt and said solvent comprises a cyclomethicone.

24. The underarm deodorant in accordance with claim 17, wherein the solvent comprises at least one of a group of substances consisting of: an antibacterial compound, purified water, guar gum, vegetable glycerin, glycerol stearate, patchouli, and sandalwood essential oil.

25. The underarm deodorant in accordance with claim 17, wherein the solvent comprises a cyclomethicone.

26. The underarm deodorant in accordance with claim 18, wherein the antiperspirant component comprises an aluminum salt.
